(19) United States
(12) Patent Application Publication
(10) Pub. No.: US 2009/0291884 A1
(43) Pub. Date: Nov. 26, 2009

(54) Proteins for use in diagnosing and treating infection and disease

(76) Inventors: Michael Agadjanyan, Huntington Beach, CA (US); Haig Keledjian, Azusa, CA (US)

Correspondence Address: BERLINER & ASSOCIATES, 555 WEST FIFTH STREET, 31ST FLOOR, LOS ANGELES, CA, 90013, US

(21) Appl. No.: 11/973,920

(22) Filed: Oct. 11, 2007

Related U.S. Application Data
(60) Provisional application No. 60/864,118, filed on Nov. 2, 2006.

(51) Int. Cl. A61K 38/17; G01N 33/53; A61P 31/18

(52) U.S. Cl. 514/12; 435/7.1

(57) ABSTRACT

The present invention describes a composition comprised on cystatin A and at least one histone used in diagnostic tools and for the treatment of diseases associated with reduced T helper cell counts such as HIV-1 infection, AIDS, ARC, multiple sclerosis, chronic fatigue syndrome, heumatoid arthritis, Alzheimer's disease, dermatitis, type 1 diabetes mellitus, colitis, inflammatory bowel disease / irritable bowel syndrome, Crohn's disease, Psoriasis, Chronic obstructive pulmonary disease, System lupus erythematosus, transplant rejection and cancer.

### FIELD OF THE INVENTION

This invention relates to the areas of immunology and virology and specifically relates to compositions comprising cystatin A and histone, which are useful as diagnostics and therapeutics for infection and disease such as human immunodeficiency virus (HIV) infection and related diseases such as acquired immunodeficiency syndrome (AIDS) and AIDS-related complex (ARC), as well as diseases associated with a decrease in T cell count.

### BACKGROUND

Bone marrow produces cells which are destined to become immune cells. These cells become lymphocytes or phagocytes. Lymphocytes are small white blood cells that bear the major responsibility for carrying out the activities of the immune system. The two major classes of lymphocytes are B cells and T cells. B cells mature in the bone (thus the term “B cells”) marrow. T cells migrate to the thymus (thus the term “T cells”) where they multiply and mature into cells capable of immune response. Upon exiting the bone marrow and thymus, both B and T cells travel widely and continuously throughout the body.

There are two types of T cells, regulatory and cytotoxic T cells, which contribute to the immune defenses in two major ways. Chief among the T cells are “helper/inducer” cells. Identifiable by the T4 cell marker, helper T cells are essential for activating B cells and other T cells as well as natural killer cells and macrophages. Cytotoxic T cells are killer cells which, for example, directly attack and rid the body of cells that have been infected by viruses or transformed by cancer.

Important phagocytes are monocytes and macrophages. Monocytes circulate in the blood, then migrate into tissues where they develop into macrophages (“big eaters”). Macrophages are found throughout the body tissues and are versatile cells that play many roles. As scavengers, they rid the body of worn-out cells and other debris. Foremost among cells that present antigen to T cells, having first digested and processed it, macrophages play a crucial role in initiating the immune response. As secretory cells, monocytes and macrophages are vital to the regulation of immune responses. They also carry receptors for lymphokines that allow them to be “activated” to pursue microbes and tumor cells.

Some diseases, such as Acquired Immunodeficiency Syndrome (AIDS,) are caused by a virus, in the case of AIDS, the human immunodeficiency virus (HIV). Such viruses destroy helper T cells and, again using AIDS as an example, is harbored in macrophages and monocytes. Entry of HIV-1 into helper T cells involves the primary receptor CD4 and co-receptors CCR5 and CXCR4. The first step in cell entry occurs when the HIV-1 glycoprotein gp120 binds to the CD4 receptors on target cells. The next step is an interaction between the HIV-1 envelope protein and the co-receptor CCR5. Once gp120 interacts with receptor and co-receptor, the HIV-1 envelope protein gp41 undergoes a conformational change and literally brings the viral membrane into close proximity with the cell membrane. Fusion of two lipid bilayers then occurs, allowing intracellular entry of the viral contents (see, for example, Nature (1997) 387:426-430).

When HIV infects a human patient, it incorporates itself into the deoxyribonucleic acid (DNA) of the immune cells and for a variable period of between 3 months to years, the patient may not exhibit any immunodeficiency symptoms and sometimes does not produce a detectable level of antibodies against AIDS. Since an initial HIV infection may not immediately lead to detectable clinical disease symptoms or a detectable level of antibodies, the term “HIV infection” as used herein encompasses both the infection and any disease resulting therefrom, the latter being termed “HIV-related diseases”. Examples of HIV-related diseases are AIDS and ARC. After the above incubation period, the HIV multiplies within the infected cell and eventually bursts the host cells which release the newly formed viruses. Since the host cells are destroyed in the process, the patient's immune system is impaired and the host is susceptible to opportunistic diseases that a human with intact immune system is not susceptible to. In human, generally the AIDS virus will multiply and the human will eventually die from severe immunodeficiency. Interestingly, only humans suffer from AIDS. When a non-human mammal, such as a rabbit, mouse, rat or cow, is injected with HIV, the animal may temporarily have some T cells destroyed. However, 14 to 21 days post-infection, the animal would mount an antibody attack and does not succumb to AIDS. Thus, there is no animal model for AIDS.

Currently, despite enormous efforts there is no cure for AIDS and the available therapeutic treatments have limited, and in some cases negligible, results.

Accurately diagnosing AIDS at an earlier stage of the disease has also been the focal point of research efforts. Currently, the commercially available diagnostic tests are generally directed to detecting the patient's antibodies against HIV. But antibody production against the virus generally does not occur until about 14 to 21 days after the time the patient is infected with AIDS. Therefore, if a patient is tested before antibody production has begun and is quantitatable; the tests will produce a false negative result. On the other hand, some of these tests may also give false positive results due to non-specific binding of the antibodies. Another means for detecting the viral infection is through nucleic acid hybridization.

Unless otherwise noted, the following is based on Stein et al. (1992) Infect. Diseases, 165: 352.

The surrogate marker that most closely correlates with the stage of HIV infection is the CD4⁺ or T helper, cell count. HIV-1 envelope glycoprotein, gp120, specifically binds to the CD4 receptor that is expressed in greatest concentration in a subset of T lymphocytes and in lower amounts on monocytes and macrophages. Cells expressing CD4 receptors are termed the “helper/inducer” subset, reflecting their role as both helper cells for B cell responses for antigens expressed on cells bearing human leukocyte antigen (HLA) class II receptors and inducer cells that cause T cells to suppress immune responses. The selective loss of CD4⁺ cells results in numerous immune defects associated with susceptibility to the opportunistic infections that are the hallmark of

The HIV core antigen p24 can be detected before the appearance of HIV antibodies. After the appearance of HIV antibodies by the screening enzyme-linked immunosorbent assay (ELISA), p24 antigenemia generally becomes undetectable, though it can occasionally persist and often will recur later in the disease. HIV-1 titers found in plasma and peripheral blood mononuclear cell cultures also fall rapidly as specific antibodies are detectable, suggesting at least a transiently effective host immune response. Markers of immune stimulation include β₂-microglobulin.

In patients followed from the time of seroconversion, CD4⁺ cell decline has been correlated with progression to AIDS. Serum levels of β₂-microglobulin and detection of p24 antigen in blood were also both independently correlated with rates of progression. Combined with CD⁴ cell counts, use of β₂-microglobulin and p24 antigen increased prognostic accuracy for progression to AIDS compared with CD4⁺ cell count alone.

It was rare, however, for seroconverters to have a consistent decline in their percentage of CD4⁺ cells over the next three years. In the interval between visits, stable or declining levels of CD4⁺ cell percentages were found in 38% of subjects, with 12% experiencing declines followed by a leveling in their rates of loss of CD⁴ cells. Overall, 62% experienced declines in their CD4⁺ cell percentage over three years of follow-up.

In a study of 306 HIV-infected seropositive homosexual men with unknown times of seroconversion, both a CD4⁺ cell count<500/μl and p24 antigen detection were predictive of AIDS within 30 months.

Increased CD8⁺ cell counts were found to be somewhat predictive of subsequent development of AIDS.

To better correlate clinical end points, such as survival and progression to AIDS, with surrogate markers of antiviral therapy effects, analysis of additional markers such as neopterin and β₂-microglobulin, among others, have been combined with the CD4 cell count and p24 antigen.

In a limited study (Jacobson (1991) BNJ, 302:73) of patients with AIDS and ARC who tolerated an anti-AIDS drug, zidovudine, and who survived for 12 weeks, the following was found.

After controlling for three factors (age, diagnosis of AIDS at baseline, log of the baseline serum neopterin concentration), the log of the CD4⁺ cell count at 8-12 weeks, but not the change over time, best predicted subsequent survival. A decrease in β₂-microglobulin concentration at 8-12 weeks significantly predicted survival and, combined with the log of the CD4⁺ cell count, provided the best predictive model. Decreases in p24 antigenemia, serum neopterin concentrations, and the Karhofsky performance status (a measure of function in routine activities) did not significantly correlate with survival on therapy.

Stein et al. (1992 Infect. Diseases 165:352) , conclude that changes in CD4⁺ cell counts and other surrogate markers may be increasingly used as the sole end point for investigations of antiretroviral activity, of a drug or therapy, in patients with early HIV infection.

Other diseases, such as type 1 diabetes mellitus, colitis and Crohn's disease, are not currently known to be caused by viral infection. But these diseases are also associated with a decrease in the number of helper T(T_{H}) cells.

The current invention, therefore, discloses a composition containing cystatin A and at least one histone for making diagnostics and therapeutics for HIV-1 infection, AIDS and ARC and other diseases associated with a decrease in helper T cell numbers.

The present invention further embodies a method for treatment of subjects having contracted, or at risk of contracting, HIV-1 infection, AIDS, ARC and other depleted T cell associated diseases by administering a composition suitable for administration to humans containing cystatin A and at least one histone.

In another embodiment of the present invention cystatin A and at least one histone are contained in a diagnostic used to identify HIV-1 infection.

A further embodiment of the present invention includes a kit that allows identification of HIV-1 infection using cystatin A and at least one histone.

Other embodiments of the present invention include methods of treatment of diseases associated with a decrease in the number of T_{H} cells (Simpson et al. (2002) Clin Exp Allergy 32:37-42; Bottini et al. (2005) Intl Arch Allergy Immunol 138:328-333), such as multiple sclerosis (Nakajima et al. (2004) European Neurology 52:162-168), chronic fatigue syndrome, rheumatoid arthritis (Leader (1998) Ann Rheum Dis 57:328-330, Alzheimer's disease, dermatitis (Feizy and Ghobadi, Dermatology Online Journal 12(3):3), type 1 diabetes mellitus (Feizy and Ghobadi, Dermatology Online Journal 12(3):3), colitis (Fort et al. (2001) J Immunol 166:2793-2800), inflammatory bowel disease/irritable bowel syndrome (Weinstock and Summers (2001) Currents Vol 2, Number 1; Fichtner-Feigl et al. (2005) J Clin Invest doi: 10.1172/JCI24792), Crohn's disease (Sato et al. (2005) Gut 54:1254-1262), Psoriasis (Simpson et al. (2002) Clin Exp Allergy 32:37-42), Chronic obstructive pulmonary disease (Bottini et al. (2005) Intl Arch Allergy Immunol 138:328-333), System lupus erythematosus, transplant rejection and cancer (Wu et al. (2005) Leukemia 19:268-274; Vujanovic et al. (2006) Cancer Gene Therapy 13:798-805) by administering a composition suitable for administration to humans containing cystatin A and at least one histone.

### BRIEF DESCRIPTION OF THE FIGURES

FIG. 1 TNP binds to HIV-1 envelope glycoproteins and human CD4 molecules. (A) 10% SDS-PAGE analysis of TNP following by Coomassie stain (Lane 1—Molecular-weight standards; Lane 2—TNP 80 μg/mL). Representative binding sensorgrams of TNP to human CD4 molecule (B), HIV-1 full-length gp41 (C) and gp120 (D) glycoproteins immobilized on a Biacore sensor chip (8 μg/mL; 1.6 μg/mL; 0.4 μg/mL).

FIG. 2 presents SDS-PAGE analysis of TNP proteins purified via binding to HIV-1 gp120 and CD4.

FIG. 3 presents representative binding activities of histone fraction H1, a heterogeneous mixture of all histone fractions, unfractionated whole histone and BSA to human CD4 and HIV-1 gp120.

### DETAILED DESCRIPTION OF THE INVENTION

1. Definitions

The following definitions are used throughout the application.

Adjuvant: This term is used to describe a substance incorporated into, associated with or administered simultaneously with antigen which potentiates the immune response, either specifically or nonspecifically.

Histone: Unless otherwise noted, this term encompasses all histone proteins including H1, H2A, H2B, H3, H4 and H5.

Suitable For Administration to Humans: This term requires that a compound or composition be nontoxic and sufficiently pure so that no further manipulation of the compound or composition is needed prior to administration to humans.

Thymus Proteins: This term describes those proteins that are exclusively produced in and found in the thymus. The term also includes proteins that are incorporated into structures or participate in physiological process occurring in all cell types. As an example, as histone and ubiquitin are considered thymus proteins while albumin and insulin are not thymus proteins.

2.The Invention

The present invention discloses a composition suitable for administration to humans containing cystatin A and at least one histone. These proteins are present in subfractions of extracts obtained from thymus and have sometimes been described as “thymus nuclear protein (TNP)” when isolated from calf thymus (see for example US 20040018639).

More particularly, the cystatin A and at least one histone have molecular weights of about 12 kD and 15 and/or 16 kD, respectively. These proteins can be isolated by conducting a size exclusion procedure on an extract from the thymus of any mammal such as calf, sheep, goat, pig, etc. using standard protocols. For example, thymus extract can be obtained using the protocol of Hand et al. (1967) Biochem. BioPhys. Res. Commun. 26:18-23; Hand et al. (1970) Experientia 26:653-655; or Moudjou et al (2001) J Gen Virol 82:2017-2024. Size exclusion chromatography has been described in, for example, Folta-Stogniew and Williams (1999) J. Biomolec. Tech. 10:51-63 and Brooks et al. (2000) Proc. Natl. Acad. Sci. 97:7064-7067.

Cystatin A and histone(s) are purified from the resulting size selected protein solution via successive binding to at least one of CD4, gp120 and gp41. Purification can be accomplished, for example, via affinity chromatography as described in Moritz et al. (1990) FEBS Lett. 275:146-50; Hecker et al. (1997) Virus Res. 49:215-223; McInerney et al. (1998) J. Virol. 72:1523-1533 and Poumbourios et al. (1992) AIDS Res. Hum. Retroviruses 8:2055-2062.

Further purification can be conducted, if necessary, to obtain a composition suitable for administration to humans. Examples of additional purification methods are hydrophobic interaction chromatography, ion exchange chromatography, mass spectrometry, isoelectric focusing, affinity chromatography, HPLC, reversed-phase chromatography and electrophoresis to name a few. These techniques are standard and well known and can be found in laboratory manuals such as Current Protocols in Molecular Biology, Ausubel et al (eds), John Wiley and Sons, New York.; Protein Purification: Principles, High Resolution Methods, and Applications, 2nd ed., 1998, Janson and Ryden (eds.) Wiley-VCH; and Protein Purification Protocols, 2nd ed., 2003, Cutler (ed.) Humana Press.

Alternatively, cystatin A and histone(s) can be purchased commercially, mixed and purified to a state suitable for administration to humans as described above. Vendors for cystatin A and histone(s) include, for example, Sigma, ProSpec-Tany TechnoGene LTD, Lab Vision Corporation, Upstate Cell Signaling Solutions and Stressgen Bioreagents, to name but a few. The ratio of cystatin A to the at least one histone can range from 0.01 weight percent (wt %): 0.99 wt % to 0.99 wt %:0.1 wt %. One preferred range is 10 wt % cystatin A to 90 wt % histone.

3. Important Characteristics of the Composition of the Invention

The composition of the current invention which contains cystatin A and at least one histone is of interest because when the composition is administered to a diseased individual, it improves health over time compared to untreated individuals, as evidenced by the results of various experiments disclosed below. In particular, individuals having received the composition of the current invention display increases in the number or TH cells compared to untreated individuals. For example, individuals treated with the composition of the current invention exhibit increases in TH cells of at least 10%, 25%, 40%, 50%, 60%, 70%, 80%, 90%, 100% or more, In addition, individuals treated with the composition of the current invention exhibit an increase in weight gain of 0.1-1 kg, 1-2 kg, 2-3 kg or more than 3 kg.

For patients suffering from a viral or retroviral infection, treatment with the composition of the current invention can effect a reduction in viral load of at least 10%, 25%, 40%, 50%, 60%, 70%, 75%, 80%, 90%, 100% or more.

Furthermore, the effects obtained by treatment with the composition of the current invention are maintained for at least 90 days, 150 days, 180 days, 240 days, 330 days, 667 days or more after conclusion of treatment.

The composition of the invention can be used directly or can be mixed with suitable adjuvants and/or carriers. Suitable adjuvants include aluminum salt adjuvants, such as aluminium phosphate or aluminium hydroxide, calcium phosphate nanoparticles (BioSante Pharmaceuticals, Inc.), ZADAXIN™, nucleotides ppGpp and pppGpp, killed Bordetella pertussis or its components, Corenybacterium derived P40 component, cholera toxin and mycobacteria whole or parts, and ISCOMs (DeVries et al., 1988; Morein et al., 199&, Lovgren ; al., 1991). The skilled artisan is familiar with carriers appropriate for pharmaceutical use or suitable for use in humans.

4.Use of the Composition of the Invention

Injections (intramuscular or subcutaneous) will be the primary route for therapeutic administration of the composition of the invention. However, intravenous delivery, delivery through catheter, or other surgical tubing may also be used. Alternative routes include tablets and the like, liquid formulations, and inhalation of lyophilized or aerosolized receptors. Liquid formulations may be utilized after reconstitution from powder formulations.

The composition may also be administered via microspheres, liposomes, other microparticulate delivery systems or sustained release formulations placed in certain tissues including blood.

The dosage of the composition administered will depend upon the properties of the formulation employed, e.g. its binding activity and in vivo plasma half-life, the concentration of the composition in the formulation, the administration route, the site and rate of dosage, the clinical tolerance of the patient involved, the pathological condition afflicting the patient and the like, as is well within the skill of the physician.

Different dosages are used during a series of sequential inoculations; the practitioner may administer an initial inoculation and then boost with relatively smaller doses of the composition.

The following is an example of a TF formulation, dosage and administration schedule. The individual is administered an intramuscular or subcutaneous injection containing 8 mg of the composition (preferably 2 ml of a formulation containing 4 mg/ml of the composition in a physiologically acceptable solution) or 57 μg of TF protein per 1 kg body weight of the patient. Each treatment course consists of 16 injections; with two injections on consecutive days per week for 8 weeks. The patient's disease condition is monitored by means described below. Three months after the last injection, if the patient is still suffering from the disease, the treatment regimen is repeated. The treatment regimen may be repeated until satisfactory result is obtained, e.g. a halt or delay in the progress of the disease, an alleviation of the disease or a cure is obtained. Preferably, the composition is formulated in an aluminum hydroxide adjuvant. For example, the final 1 ml of the final composition formulation can contain: 4 mg of the composition, 0.016 M AlPO₄ (or 0.5 mg Al³⁺), 0.14 M NaCl, 0.004 M CH₃COONa, 0.004 M KCl, pH 6.2.

Alternatively, the individual may be inoculated five months later, more preferably six months to two years later, and even more a preferably eight months to one year later to enhance the patient's “immune memory”. See Anderson et al., Infectious Diseases, 160 (6):960-969 (1989). Generally, infrequent immunizations with the composition spaced at relatively long intervals is more preferred than frequent immunizations in eliciting maximum immune responses.

The composition of the invention can be administered in various ways and to different classes of recipients.

The composition of the invention can be administered in combination with other antigens in a single inoculation “cocktail”. The composition can also be administered as a series of inoculations administered over time. Such a series may include inoculation with the same or different preparations of antigens or other vaccines.

The adequacy of the treatment parameters chosen, e.g. dose, schedule, adjuvant choice and the like, is determined by taking aliquots of serum from the patient and assaying for antibody and/or T cell titers during the course of the treatment program. T cell titer may be monitored by conventional methods. For example, T lymphocytes can be detected by E-rosette formation as described in Bach, F., Contemporary Topics in Immunology, Vol. 2: Thymus Dependency, p. 189, Plenum Press, New York, 1973; Hoffmnan, T. & Kunkel, H. G., and Kaplan, M. E., et al., both papers are in In vitro Methods in Cell Mediated and Tumor Immunity, B. R. Bloom & R. David eds., Academic Press, New York (1976). For example, the amount of T cell rosette formation may be assayed after the third but before the tenth week of treatment. An over sixty-five percent rosette formation indicates a good cell mediated immune response in the patient.

In addition, the clinical condition of the patient can be monitored for the desired effect, e.g. increases in T cell count and/or weight gain. If inadequate effect is achieved then the patient can be boosted with further treatment and the treatment parameters can be modified, e.g. to potentiate the immune response, such as by increasing the amount of the composition of the invention and/or adjuvant, complexing cystatin A and/or the at least one histone with a carrier or conjugating them to an immunogenic protein, or varying the route of administration.

The composition may optionally be administered along with other pharmacologic agents used to treat the disease contracted by the individual such as HIV infection, AIDS and ARC. Examples of these pharmacologic agents are: AZT, antibiotics, immunomodulators such as interferon, anti-inflammatory agents and anti-tumor agents. Other diseases, such as multiple sclerosis, rheumatoid arthritis, type I diabetes mellitus and inflammatory bowel syndrome are associated with other pharmacologic agents. Identifying the appropriate pharmacologic agents is well within the skill of the physician.

Diagnostic Devices for Detecting Infection and/or Disease

Another aspect of the invention presents diagnostic devices useful for in vitro detection of infection and/or disease.

Having described the invention, the following examples are presented to illustrate the invention, and are not to be construed as limiting the scope of the invention.

5. Experiments Confirming the Usefulness of the Composition of the Invention

### EXAMPLE 1

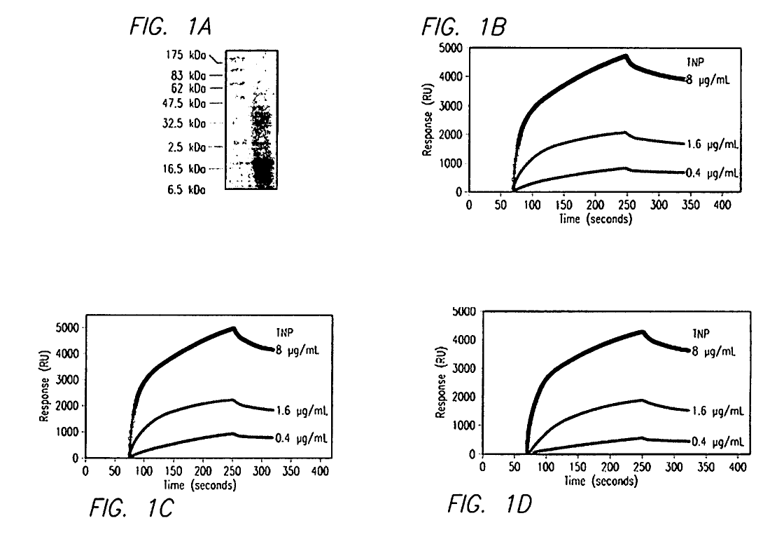

Thymus proteins were isolated from freshly sacrificed calf thymus according to US 20040018639. The protein concentration was determined by the Bradford assay with bovine serum albumin (Sigma, Cat. No A-3912) as the calibration standard. The purity of the samples was analyzed by SDS-Polyacrylamide gel electrophoresis (SDS-PAGE) using 10% and/or 15% polyacrylamide gels. The resolved proteins were visualized by Coomassie brilliant blue-R250 and/or Silver Staining (BioRad, Cat #161-0443) according to the manufacturer's protocol. Molecular weights of proteins bands were estimated by comparing their relative mobility to those of marker proteins of known molecular weights (BioRad, Cat. #161-0314), run on the same gel (FIG. 1A).

Binding studies were performed on the BIAcore 2000 (Biacore, Sweden). Recombinant human CD4 (Progenics, Cat. #PRO 1008-1), recombinant HIV-1 gp120 (NIH AIDS Research & Reference Reagent Program, #4961) and gp41 (546-682 aa) were immobilized to the surface of biosensor chip (CM5) via an amine coupling of the appropriate protein to carboxyl groups in the dextran matrix of the chip. Serial dilutions of the crude sample in the running buffer containing 10 mM HEPES, 150 mM NaCl, 0.05% surfactant P20, pH 7.4 were injected at 5 μl/min over each immobilized target and the kinetics of binding/dissociation was measured as change of the SPR signal (in resonance units—RU). Each injection was followed by a regeneration step of 30-sec pulse of 1M NaCl, 50 mM NaOH. Fitting of experimental data was done with BIAevaluation 3.0 software. The crude protein strongly bound to CD4 molecules (FIG. 1B) and to gp41 and gp120 of HIV-1 (FIGS. 1C and 1D, respectively), but not to BSA.

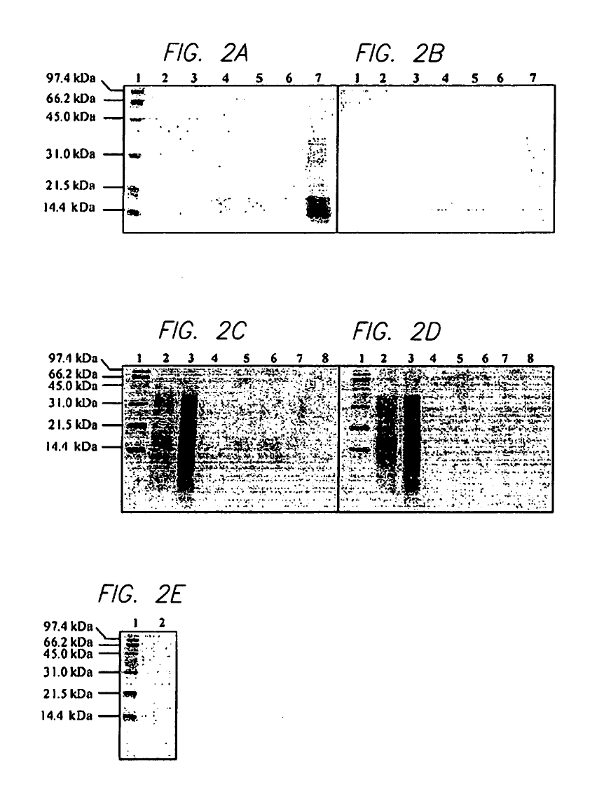

Protein fractions from the isolated thymus protein sample were purified using an affinity chromatography column (MicroLink™ Protein Coupling Kit, Pierce, Cat. #20475) according to the manufacture's instructions. Briefly, 0.2 mg of recombinant human CD4 (Progenics, Cat. #PRO 1008-1) or recombinant HIV-1 gp120 (NIH AIDS Research & Reference Reagent Program, #4961), or irrelevant antigen (amyloid beta peptide) were immobilized on an AminoLink coupling gel and the remaining active binding sites were blocked with IM Tris•HCl, 0.05% NaN₃. 1 mL of crude thymus protein sample was incubated with the immobilized protein to form an immune complex. The gel-bound complex was then washed to remove irrelevant material. Proteins specifically bound to CD4 or gp120 were eluted with primary amines containing solution (pH 2.8) and neutralized. Eluted fractions were analyzed by 15% SDS-PAGE followed by Coomassie brilliant blue-R250 and/or silver staining (FIGS. 2A and 2B, respectively) and the concentration was determined by Bradford protein assay. Molecular sizes of these bands were around 14-17 kDa.

Specificity of these proteins was confirmed by purifying the same sample using two amino link columns, one coupled with gp120 and another one with human amyloid beta peptide, and running different fractions of the eluted proteins on a 15% SDS-PAGE gel. Three slender bands were detected representing low molecular weight proteins specific to gp120 in fractions #2, #3, and #4 eluted from the column with gp120 (FIG. 2C), while no protein was found in any fractions eluted from the column with amyloid beta protein (FIG. 2D). Fractions #2-4 eluted from the gp120 column were passed through another amino link column coupled with CD4. All three proteins that bound to gp120 were also specific to CD4 molecules, and 14-17 kDa bands detected in a 15% SDS-PAGE gel (FIG. 2E).

### EXAMPLE 2

Sequence analysis of the three bands with approximate molecular weights of 16,000; 15,000 and 12,000 Daltons was performed at the Molecular Structure Facility at the University of California, Davis by de novo sequencing using tandem mass spectrometry. Protein analysis was performed using a Finnigan LCQ Deca XP Plus (San Jose, Calif.) coupled directly to an LC column. The Sequest analysis software (Bioworks v. 3.1) was used to identify the peptide sequences in a human or bovine protein database that best match the observed MS/MS spectra.

The results from the bovine database identified the 16 kDa protein as histone H1.1 or H2B. Analysis also indicates that the 15 kDa and 12 kDA proteins likely represent bovine H1.1 sequence (50.5% and 48.6% sequence coverage, respectively). In addition to these analyses the sequences were also compared to the human database. Again, the 16 kDa protein likely represents human histone H2.B (42.1% coverage), although the sequence of this protein has 24.5% identity with amino aid sequence of human Cystatin A as well. Interestingly, the 15 kDa protein also showed 42.9% identity to cystatin A while the 12 kDa protein showed 61.2% identity. Of note, these molecules also had about 24% identical amino acids sequences with HI histone family.

### EXAMPLE 3

The identity of histones and cystatin A was confirmed by directly demonstrating binding of these proteins to HIV1 gp120 and human CD4 molecules. Binding studies were performed on the BIAcore 2000 (Biacore, Sweden). Recombinant human CD4 (Progenics, Cat. #PRO 1008-1), recombinant HIV-1 gp120 (NIH AIDS Research & Reference Reagent Program, #4961) and gp41 (546-682 aa) were immobilized to the surface of biosensor chip (CM5) via an amine coupling of the appropriate protein to carboxyl groups in the dextran matrix of the chip. Serial dilutions of the crude sample in the running buffer containing 10 mM HEPES, 150 mM NaCl, 0.05% surfactant P20, pH 7.4 were injected at 5 μl/min over each immobilized target and the kinetics of binding/dissociation was measured as change of the SPR signal (in resonance units—RU). Each injection was followed by a regeneration step of 30-sec pulse of 1M NaCl, 50 mM NaOH. Fitting of experimental data was done with BIAevaluation 3.0 software.

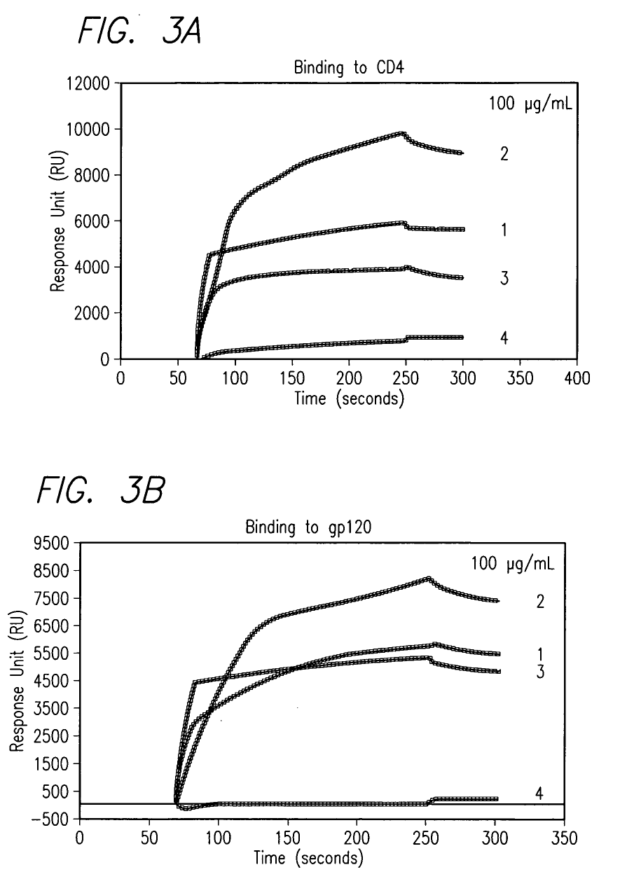

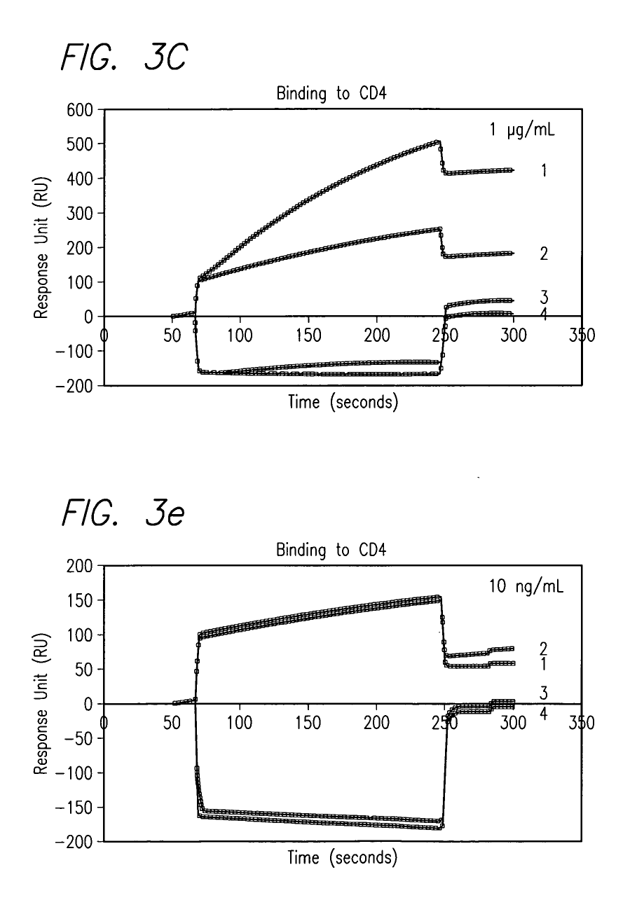

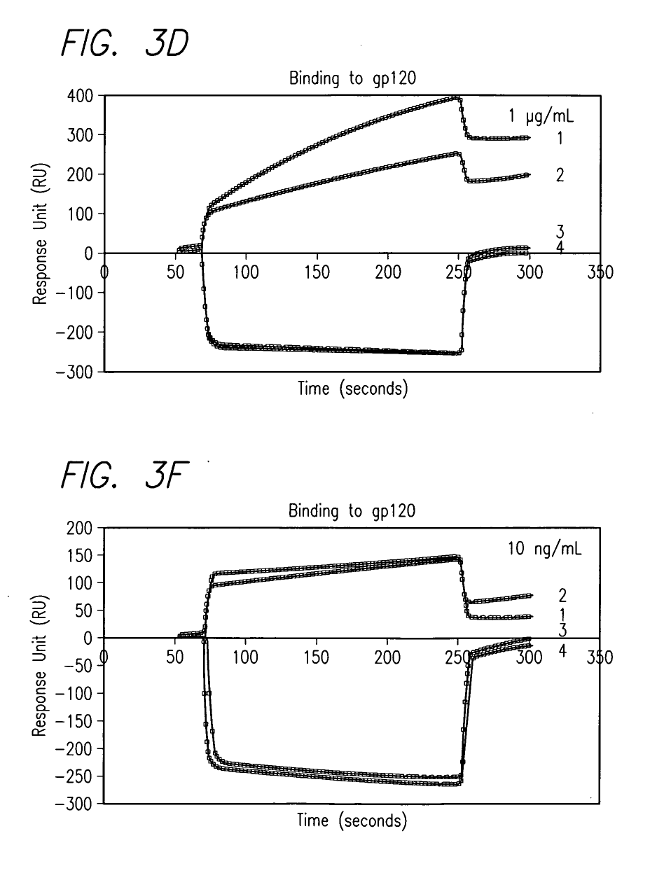

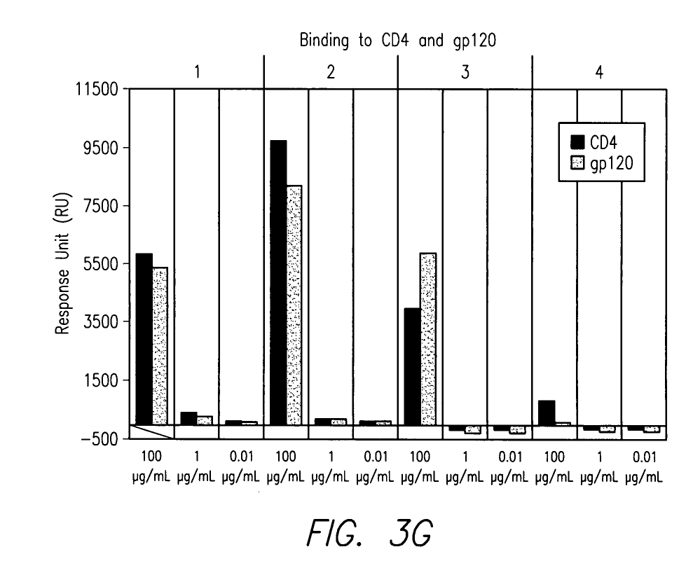

Four out of five histones bound to gp120 and CD4 molecules very well (FIGS. 3A and B). However, the affinity of binding to gp120 was significantly higher than that for CD4.

### EXAMPLE 4

The binding affinity of the cystatin A and histone components of the composition are determined using any standard protocol, such as isothermal titration calorimetry (Velazquez-Campoy and Freire (2006) Nature Protocols 1 :186-191 ;Sigurskjold (2000) Anal Biochem 277:260-266; Wiseman et al. (1989) Anal. Biochem 179:131-137; which are incorporated in their entirety by reference). Alternatively, the binding affinities are determined using Biacore technology.

We claim:

1. A composition comprising
(a) a cystatin A protein; and
(b) at least one histone protein,
wherein said composition is free of other thymus proteins and suitable for administration to humans, optionally with a pharmaceutically acceptable adjuvant or carrier.

2. The composition of claim 1, wherein said at least one histone protein is a histone H1.

3. The composition of claim 1, wherein said at least one histone protein is a histone H2A.

4. The composition of claim 1, wherein said at least one histone protein is a histone H2B.

5. The composition of claim 1 wherein two histone proteins are present.

6. The composition of claim 5, wherein said two histone proteins are a histone HI and a histone H2.

7. The composition of claim 1, wherein said composition has a binding affinity for gp120 of at least 5000 RU.

8. The composition of claim 1, wherein said composition has a binding affinity for gp41 of at least 5000 RU.

9. The composition of claim 1, wherein said composition has a binding affinity for CD4 of at least 5000 RU.

10. The composition of claim 1, wherein said adjuvant is aluminum hydroxide or aluminum phosphate.

11. The composition of claim 1, wherein said adjuvant is calcium phosphate.

12. The composition of claim 1, wherein said adjuvant is selected from the group consisting of monophosphoryl lipid A, ISCOMs with Quil-A, and Syntex adjuvant formulations (SAFs) containing the threonyl derivative or muramyl dipeptide.

13. A composition comprising
(a) a cystatin A protein; and
(b) at least one histone protein,
wherein said composition is free of other thymus proteins and suitable for administration to humans, and at least one of said cystatin A protein and said at least one histone protein is complexed to at least one member selected from the group consisting of CD4, gp120 and gp41, optionally with a pharmaceutically acceptable adjuvant and/or carrier.

14. The composition of claim 1, wherein said composition has a binding affinity for gp120 of at least 5000 RU.

15. The composition of claim 1, wherein said composition has a binding affinity for gp41 of at least 5000 RU.

16. The composition of claim 1, wherein said composition has a binding affinity for CD4 of at least 5000 RU.

17. The composition of claim 1, wherein said adjuvant is aluminum hydroxide or aluminum phosphate.

18. The composition of claim 1, wherein said adjuvant is calcium phosphate.

19. The composition of claim 1, wherein said adjuvant is selected from the group consisting of monophosphoryl lipid A, ISCOMs with Quil-A, and Syntex adjuvant formulations (SAFs) containing the threonyl derivative or muramyl dipeptide.

20. A method of treatment for AIDS (HIV-1 infection) or individuals at risk of acquiring AIDS comprising administering to a subject in need thereof a composition comprising
(a) a cystatin A protein; and
(b) at least one histone protein,
wherein said composition is free of other thymus proteins and suitable for administration to humans, optionally with a pharmaceutically acceptable adjuvant or carrier.

21. A method of treatment for AIDS (HIV-1 infection) or individuals at risk of acquiring AIDS comprising administering to a subject in need thereof a composition to humans comprising
(a) a cystatin A protein; and
(b) at least one histone protein,
wherein said composition is free of other thymus proteins and suitable for administration to humans, and at least one of said cystatin A protein and said at least one histone protein is complexed to at least one member selected from the group consisting of CD4, gp120 and gp41, optionally with a pharmaceutically acceptable adjuvant and/or carrier.

22. The method according to claim 20 or 21, wherein said administration occurs over a period of eight weeks.

23. The method according to claim 22, wherein said administration is bi-weekly.

24. The method according to claim 23, wherein said bi-weekly administration is on consecutive days.

25. The method according to claim 20 or 21, wherein said administration is at least one of oral, parenteral, subcutaneous, intravenous, intramuscular and mucosal administration.

26. The method according to claim 20 or 21, wherein said composition has a binding affinity for gp120 of at least 5000 RU.

27. The method of claim 20 or 21, wherein said composition has a binding affinity for gp41 of at least 5000 RU.

28. The method of claim 20 or 21, wherein said composition has a binding affinity for CD4 of at least 5000 RU.

29. The method of claim 20 or 21, wherein said adjuvant is aluminum hydroxide or aluminum phosphate.

30. The method of claim 20 or 21, wherein said adjuvant is calcium phosphate.

31. The method of claim 20 or 21, wherein said adjuvant is selected from the group consisting of aluminum salt adjuvants, such as aluminium phosphate or aluminium hydroxide, calcium phosphate nanoparticles (BioSante Pharmaceuticals, Inc.), ZADAXIN™, nucleotides ppgpp and pppGpp, killed Bordetella pertussis or its components, Corenybacterium derived P40 component, killed cholera toxin or its parts and killedmycobacteria or its parts.

32. A method for diagnosing HIV-1 infection (AIDS) comprising
(a) collecting a blood, serum or plasma sample from a subject;
(b) mixing said sample with a composition comprising
(i) a cystatin A protein; and
(ii) at least one histone protein; and
(c) identifying a complex of said composition bound to any one of CD4, gp120 and gp41,
wherein said complex is indicative of HIV-1 infection.

33. The method of claim 32, wherein said complex is identified by electrophoresis.

34. The method of claim 32, wherein said complex is identified by chromatography.

35. The method of claim 32, wherein said complex is identified by HPLC.

36. The method of claim 32, wherein said complex is identified by an immunological reaction.

37. A kit for detection of HIV infection comprising comprising
(a) a cystatin A protein;
(b) at least one histone protein; and
(c) a device for identifying at least one complex of said cystatin A protein and said at least one histone protein with CD4, gp120 or gp41.

38. A method of treatment for disease associated with a decrease in the number of TH cells comprising administering to a subject in need thereof a composition comprising
(a) a cystatin A protein; and
(b) at least one histone protein,
wherein said composition is free of other thymus proteins and suitable for administration to humans, optionally with a pharmaceutically acceptable adjuvant or carrier.

39. The method according to claim 38, wherein said disease is selected from the group consisting of multiple sclerosis, chronic fatigue syndrome, heumatoid arthritis, Alzheimer's disease, dermatitis, type 1 diabetes mellitus, colitis, inflammatory bowel disease / irritable bowel syndrome, Crohn's disease, Psoriasis, Chronic obstructive pulmonary disease, System lupus erythematosus, transplant rejection and cancer.
